(19) United States
(12) Patent Application Publication
(10) Pub. No.: US 2007/0009594 A1
(43) Pub. Date: Jan. 11, 2007

(54) Compositions and methods for treatment of cycle-related symptoms

(75) Inventors: Gary Sondermann Grubb, Newtown Square, PA (US); Ginger Dale Constantine, Malvern, PA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Wyeth, Madison, NJ (US)

(21) Appl. No.: 11/475,486

(22) Filed: Jun. 26, 2006

Related U.S. Application Data
(60) Provisional application No. 60/695,077, filed on Jun. 28, 2005.

(51) Int. Cl. A61K 31/56; A61K 9/20

(52) U.S. Cl. 424/464; 514/170

(57) ABSTRACT

Methods are provided for treating cycle-related symptoms through administration of at least one progestin and at least one estrogen to a female subject.

### CROSS-REFERENCE TO RELATED APPLICATIONS

This application claims the benefit of U.S. Provisional Application No. 60/695,077, filed Jun. 28, 2005, which is incorporated herein by reference.

### FIELD OF THE INVENTION

In one aspect, the present invention relates to methods for treating cycle-related symptoms through administration of at least one progestin and at least one estrogen to a female subject.

### BACKGROUND OF THE INVENTION

The term “cycle-related symptoms” refers to physical and psychological symptoms associated with a woman's menstrual cycle arising in the luteal phase of the menstrual cycle. It has been reported that most women report experiencing cycle-related symptoms. The symptoms generally disappear soon after the onset of menstruation, and the patient has markedly reduced or no symptoms during the rest of the follicular phase. The cyclical occurrence of the symptoms is the key characteristic of cycle-related symptoms.

Cycle-related symptoms occur in about 95% of women with their menstrual cycles. About one-third of those women experience moderate to severe cycle-related symptoms. Women vary in the number, type, severity, and pattern of these cycle-related symptoms that occur before menstruation. One thing common to all the types of cyclic-related symptoms is the decrease or elimination of the symptoms in the two weeks after menstruation up to the time of ovulation.

The use of extended oral contraceptive for extended cycles (i.e., greater than consecutive 21 days of active drug) to reduce cycle-related symptoms with tolerable irregular bleeding has shown variable success. Extension of active pill use to produce cycles varying from 42 to 84 days has been studied in small populations, with different oral contraceptive formulations, and with variable success regarding cycle control. Tonkelaar and Oddens, Contraception, 59: 357-362, 1999; U.S. patent application Ser. No. 2003/0139381. Extending cyclical oral contraceptive use from 21 to 42 days reduced bleeding and need for hygiene products. Miller and Hughes, Obstet. Gynecol., 101: 653-661, 2003. A need exists in the art for an improved method for administering an oral contraceptive to women to relieve cycle-related symptoms.

### SUMMARY OF THE INVENTION

In one aspect, the present invention provides methods for treating a female subject having cycle-related symptoms. Certain methods according to the invention comprise administering an effective amount of at least one progestin and at least one estrogen to said female subject, wherein said effective amount is administered daily for at least about 100 days. The female subject can have cycle-related symptoms and said effective amount can be effective for treating cycle-related symptoms. The female subject can have cycle-related symptoms, for example, dysmenorrhea or moderate to severe cycle-related symptoms, and said effective amount can be effective for treating cycle-related symptoms of dysmenorrhea or other physical and psychological cycle-related symptoms. Preferred among such methods are those that comprise administering an effective amount of at least one progestin and at least one estrogen to the female subject. Effect dose refers to the combined amount of steroid in a daily dosage unit taking into account the potency of a given steroid. The effect dose of a given steroid can be determined by one skilled in the art. In certain embodiments, at least about 4 μg of the at least one progestin (preferably from about 60 to about 120 μg of levonorgestrel (LNG), or more preferably about 90 μg) and/or at least about 1 μg of the at least one estrogen (or preferably from about 15 to about 25 μg of ethinyl estradiol (EE), or more preferably about 20 μg) is administered.

Also provided are methods for treating a female subject having cycle-related symptoms that comprise administering at least one progestin and at least one estrogen to a female subject daily for at least about 100 days. Preferred among such methods are those that involve daily administration for at least about 4 months, for at least about 6 months, more preferably at least about 9 months, or even more preferably for at least about 12 months. In certain methods, the female subject has cycle-related symptoms and the at least one progestin and at least one estrogen are administered in an amount effective for the treatment thereof. In still further methods, at least one progestin and at least one estrogen are administered in an amount effective for contraception.

The invention also provides kits for treating a female subject having cycle-related symptoms, comprising at least about 100 dosage forms that individually comprise at least one progestin and at least one estrogen. In preferred kits, the dosage forms comprises about 90 μg of levonorgestrel (LNG) or the at least one progestin of equivalent potencies and/or about 20 μg of ethinyl estradiol (EE) or the at least one estrogen of equivalent potencies. The kits can take the form of, for example, blister packs or other suitable dosage form arrays, and can include at least about 100 such dosage forms, at least about 185 such dosage forms, preferably at least about 275 such dosage forms, or more preferably at least about 365 such dosage forms.

### BRIEF DESCRIPTION OF THE DRAWINGS

FIG. 1 shows the 17-item Penn Daily Symptom Report (DSR) and premenstrual total score for the moderate to severe cycle-related symptoms subgroup of the cycle-related symptoms study (CRSS).

FIG. 2 shows 17-item Penn Daily Symptom Report (DSR) postmenstrual subscale score for the moderate to severe cycle-related symptoms subgroup of the cycle-related symptoms study (CRSS).

FIG. 3 shows Endicott Work Productivity Scale (EWPS) total score for the moderate to severe cycle-related symptoms subgroup of the cycle-related symptoms substudy.

### DETAILED DESCRIPTION OF ILLUSTRATIVE EMBODIMENTS

Certain methods of the invention involve treating female subjects for cycle-related symptoms associated with the menstrual cycle. As used herein, the term “treating” or “treatment” refers to any indicia of success in amelioration of an injury, pathology, or condition, including any objective or subjective parameter such as abatement; inhibition; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective parameters or subjective parameters, e.g., symptom scores and quality of life evaluations; including the results of a physical examination, neurological examination, and/or psychiatric evaluation. Treating or treatment of any condition disclosed herein includes preventing the onset of symptoms in a subject that may be predisposed to the condition but does not yet experience or exhibit symptoms of the condition (prophylactic treatment), or inhibiting the symptoms of the condition (slowing or arresting its development). Accordingly, the term “treating” includes the administration of compounds or agents to a subject to prevent or delay, to alleviate, or to arrest or inhibit development of the symptoms or conditions associated with the condition. A skilled medical practitioner will know how to use standard methods to determine whether and to what extent a patient has cycle-related symptoms. Such a determination can be made before administration of an effective amount of progestin and estrogen and/or after administration.

The term “cycle-related symptoms” refers to psychological symptoms (for example, mood change, irritability, anxiety, lack of concentration, or decrease in sexual desire) and physical symptoms (for example, dysmenorrhea, breast tenderness, bloating, fatigue, or food cravings) associated with a woman's menstrual cycle. Cycle-related symptoms occur after ovulation but before menses and usually terminate at the start of the menstrual period or shortly thereafter. Cycle-related symptoms include, but are not limited to, dysmenorrhea and other physical and psychological cycle-related symptoms.

The term “dysmenorrhea” refers to painful uterine cramping with menses. Women with dysmenorrhea may experience nausea, vomiting, diarrhea, headaches, weakness, and/or fainting. Symptoms may vary in severity from cycle to cycle, but generally continue throughout the reproductive years. Dysmenorrhea can be an incapacitating problem, causing significant disruption in a woman's life each month.

An effective treatment for cycle-related symptoms can be determined by administering varying amounts of progestin and estrogen, conducting a cycle-related symptom study (CRSS), and measuring a reduction in cycle-related symptoms. A clinical study can evaluate cycle-related symptoms among subgroups of subjects who report symptoms of: 1) dysmenorrhea; or 2) other physical and psychological cycle-related symptoms. Various measurement scales can be used to quantify cycle-related symptoms in women. For example, a measurement of cycle-related symptoms in women can be determined by factors of the Penn Daily Symptom Report (DSR) which includes 17 items. See, for example, Freeman et al., Psychiatry Research 65: 97-106, 1996, incorporated herein by reference in its entirety. These factors can be further analyzed and subdivided into four factor subscales: 1) mood (i.e., anxiety, irritability, depression, nervous tension, mood swing, and feeling out of control); 2) behavioral symptoms (i.e., poor coordination, insomnia, confusion, headache, crying, and fatigue); 3) pain (i.e., aches, cramps, and breast tenderness); and 4) physical (i.e., food cravings, swelling). As a further example, a measurement of the effect of cycle-related symptoms on work productivity can be determined by the Endicott Work Productivity Scale. See, for example, Endicott and Nes, Psychopharmacology Bulletin 33: 13-16, 1997, incorporated herein by reference in its entirety.

Preferred methods for treating or diminishing cycle-related symptoms involve administering an effective amount of at least one progestin and at least one estrogen to a female subject. The term “progestin,” as used herein, refers to any progestationally active compound, i.e., any compound that binds to and activates any progesterone receptor. Representative progestins include progesterone synthetic derivatives such as, for example, 17-hydroxy progesterone esters, 19-nor-17-hydroxy progesterone esters, 17α-ethinyltestosterone and derivatives thereof, 17α-ethinyl-19-nor-testosterone and derivatives thereof, norethindrone, norethindrone acetate, ethynodiol diacetate, dydrogesterone, medroxy-progesterone acetate, norethynodrel, allylestrenol, lynoestrenol, fuingestanol acetate, medrogestone, norgestrienone, dimethiderome, ethisterone, cyproterone acetate, levonorgestrel, dl-norgestrel, d-17α-acetoxy-13β-ethyl-17α-a-ethinyl-gon-4-en-3-one oxime, gestodene, desogestrel, etonorgestrel, norgestimate and norelgestromin. Other compounds with progestational activity used in oral contraceptives include chlormadione, dienogest, and drospirenone. One preferred progestin is levonorgestrel.

The term “estrogen,” as used herein, refers to a group of synthetic or natural estrogens, including steroidal and nonsteroidal estrogens. The natural estrogens can be mammalian-derived or plant-derived. In humans, estrogens are formed in the ovary, possibly the adrenal cortex, the testis, and the fetoplacental unit and have various functions in both sexes. Estrogen is included within a class of ovulation inhibitors to prevent breakthrough (mid-cycle) bleeding during the ovulation cycle. The ring system of an estrogen is estrane, an 18-carbon tetracyclic hydrocarbon nucleus that is the parent structure of the estrogenic steroids. Estrogens typically have an aromatic A ring with a phenolic 3-OH group and an oxygen function on C17. Estrogens are defined as any compound that binds to and activates any estrogen receptor. The synthetic estrogens can be for example, ethinyl estradiol, ethynodiol diacetate, mestranol and quinestranol. Particularly of interest are 17α-ethinyl estradiol and esters and ethers thereof. One preferred estrogen is 17α-ethinyl estradiol. The natural estrogens can include, for example, conjugated equine estrogens, esterified estrogens, 17β-estradiol, estradiol valerate, estrone, piperazine estrone sulphate, estriol, estriol succinate and polyestrol phosphate. Other useable estrogens include the esters of estradiol, estrone and ethinyl estradiol such as the acetate, sulfate, valerate or benzoate, conjugated equine estrogens, agonist estrogens, and selective estrogen receptor modulators.

Preferred methods for treating or diminishing cycle-related symptoms involve administering an effective amount of at least one progestin and at least one estrogen administered in a continuous and uninterrupted regimen, i.e., continuous use, to a female subject and effectively reducing cycle-related symptoms typically associated with menses. For example, in a group of women with dysmenorrhea, the continuous-use regimen of at least one progestin and at least one estrogen was highly effective in significantly reducing dysmenorrhea over the 3-month treatment period. For example, in a group of women with moderate to severe cycle-related symptoms, the continuous-use regimen of at least one progestin and at least one estrogen was effective in achieving a significant reduction in all 17 moderate to severe cycle-related symptoms assessed over the 3-month treatment period.

The progestins and estrogens of the invention can be administered in any amount effective to treat cycle-related symptoms, and/or to achieve contraception. In preferred embodiments, at least about 4 μg of at least one progestin, for example, levonorgestrel (preferably from about 4 to about 120 μg, more preferably from about 60 to about 110 μg, or more preferably about 90 μg) and at least about 1 μg of at least one estrogen, for example, ethinyl estradiol (preferably from about 1 to about 25 μg, more preferably from about 15 to about 25 μg, or more preferably about 20 μg) is administered. It is preferred that the progestin dosage be not greater than 120 μg per day (when levonorgestrel is used), and that the estrogen dosage be not greater than 20 μg per day (when ethinyl estradiol is used). It is also preferred that the progestin and estrogen be administered at a constant, or at least relatively constant, daily dosage.

Although administration of ethinyl estradiol at a dosage of approximately 20 μg per day and levonorgestrel at a dosage of approximately 90 μg per day is preferred, one can use at least about 1 μg of ethinyl estradiol, (preferably from about 1 to about 25 μg, more preferably from about 15 to about 25 μg, or more preferably about 20 μg), and at least about 4 μg of levonorgestrel (preferably from about 4 to about 120 μg, more preferably from about 60 to about 110 μg, or more preferably about 90 μg). Other estrogens and progestins vary in potency from ethinyl estradiol and levonorgestrel, respectively. To the extent that other estrogens are used, either alone or in combination with ethinyl estradiol, it is preferred that the amount of estrogen used correspond to an equivalent pharmacologic potency to that of the stated amounts of ethinyl estradiol. Similarly, to the extent that other progestins are used, either alone or in combination with levonorgestrel, it is preferred that the amount of progestin used correspond to an equivalent pharmacologic potency to that of the stated amounts of levonorgestrel. The correlations in potency between the various estrogens and progestins are generally known to those skilled in the art, see, e.g., European Patent Application No.0 253 607; U.S. application Ser. No. 2003/0139381, each incorporated herein by reference in their entirety and for all purposes.

The methods for treating or diminishing cycle-related symptoms preferably involve administering progestin and estrogen daily for at least about 100 days. In certain embodiments, administration is daily for at least about 4 months, daily for at least about 6 months, daily for at least about 9 months, and/or daily for at least about 12 months. Certain methods of the invention involves administering estrogen and progestin, preferably in uniform dosages, for 28 consecutive days. The 28-day treatment cycles are continued for multiple cycles to provide a constant dosage of estrogen and progestin for up to 6 months, up to 12 months, up to 18 months, up to 24 months or longer. In preferred embodiments, women are administered an oral contraceptive on days 1 through 28 of the menstrual cycle containing 90 μg levonorgestrel and 20 μg ethinyl estradiol per day. Thus, with a 28-day treatment cycle, there are about 13 treatment cycles per year, thus eliminating all menstrual cycles in the year. The treatment regimen can be continued for an extended administration period, for example, one year or longer, or two years or longer. There is no limit to the amount of time, as long as the woman may potentially have menstrual cycles.

The formulations of the invention can be administered orally, parenterally, sublingually, subdermally, transdermally, topically, intravaginally, intranasally or buccally in a variety of suitable dosage forms. The method of administration depends on the types of estrogens and progestins used, as well as the amounts per unit dosage. Pharmaceutical formulations or preparations containing the formulations of the invention and a suitable carrier can be solid dosage forms which includes tablets, dragees, capsules, cachets, pellets, pills, powders or granules; topical dosage forms which include solutions, powders, fluid emulsions, fluid suspensions, semi-solids, ointments, pastes, creams, gels or jellies, foams and controlled release depot entities; transdermals, vaginal rings, buccal formulations; and parenteral dosage forms which includes solutions, suspensions, emulsions or dry powder comprising an effective amount of estrogen and progestin as taught in this invention. “Depot” or “drug depot” refers to a reservoir containing a composition that is implanted into, or in some fashion connected to a patient such that the compound is delivered to the patient. The depot may or may not regulate the administration of the compound.

Pharmaceutically acceptable carriers are determined in part by the particular composition being administered, as well as by the particular method used to administer the composition. Accordingly, there is a wide variety of suitable formulations of pharmaceutical compositions for administering the hormonal contraceptive product. It is known in the art that active ingredients can be contained in such formulations in addition to pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. See, e.g., Remington's Pharmaceutical Sciences, Mack Publishing Co., Easton, Pa. 18^{th} ed., 1990; Modern Pharmaceutics, Banker & Rhodes, Marcel Dekker, Inc., 1979; or Goodman & Gilman's The Pharmaceutical Basis of Therapeutics, 6th Edition, MacMillan Publishing Co., New York, 1980, each incorporated herein by reference in their entirety and for all purposes. The pharmaceutical compositions are generally formulated as sterile, substantially isotonic and in full compliance with all Good Manufacturing Practice (GMP) regulations of the U.S. Food and Drug Administration.

Generally speaking, the formulations are prepared according to conventionally known procedures in accordance with the method of administration. Thus, the active ingredients are prepared according to known methods in a pharmaceutically acceptable form for administration. These ingredients, in their required quantities are combined with the appropriate pharmaceutical carriers such as additives, vehicles and/or flavor ameliorating substances. These substances can be referred to as diluents, binders and lubricants. Gums, starches and sugars are also common terms. Typical of these types of substances or excipients are pharmaceutical grades of mannitol, lactose starch, magnesium stearate, sodium saccharin, talcum, cellulose, glucose, sucrose, magnesium carbonate and the like. The active ingredient(s) can comprise from about 0.01% by weight to about 99.99% by weight of the total formulation and the remainder comprises the pharmaceutically acceptable carrier. The percentage of active ingredient(s) can vary according to the delivery system or method of administration and is chosen in accordance with conventional methods known in the art.

Most estrogens and progestins are orally active and that route of administration (preferably in tablet or capsule form) is therefore preferred. Pharmaceutical dosage forms for oral use can be obtained through combination of the compounds of the present invention with a solid excipient, optionally grinding a resulting mixture, and processing the mixture of granules, after adding suitable additional compounds, if desired, to obtain tablets or cores. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Methods for transdermal administration, including the associated manufacturing methods, are well known in the art. In this connection, reference may be had to U.S. Pat. Nos. 4,752,478, 4,685,911, 4,438,139 and 4,291,014, each incorporated herein by reference in their entirety and for all purposes.

Dosage forms according to the invention can be placed in an appropriate package and labeled for treatment. Such packages (whether in the form of blister packs, tablet dispensers, or the like) are referred to herein as kits, typically include the daily dosages arranged for proper sequential administration. Preferred kits contain multiple dosage forms in a synchronized, fixed sequence, wherein the sequence or arrangement thereof corresponds to the stages of daily administration. For example, dosage forms can be provided in kit form containing about 18 to about 28 tablets for a 28-day regimen, preferably about 21 to about 28 tablets. These tablets are intended for ingestion on successive days. For example, dosage forms can be provided in kit form containing about 28 to about 59 tablets for three or more 28-day regimens, preferably about 51 to about 59 tablets. These tablets are intended for ingestion on successive days. For a more long-term regimen, the dosage forms can be provided in kit form containing at least about 60 tablets, and preferably at least about 81 to 89 tablets, and up to 110 tablets, intended for ingestion on successive days. Preferably administration is daily for at least 100 days. Daily administration for at least 168 days, for at least 336 days, or for a year or longer can also be effected. For administration of multiple dosage forms from a kit, the provided labeling will typically include, for example, instructions concerning the amount, frequency and method of administration of each dosage form. Preferred kits are those that include at least 100 dosage forms that individually include at least one progestin and at least one estrogen. Such kits can, in certain embodiments, include at least about 185 of the dosage forms, at least about 275 of the dosage forms, and/or at least about 365 of the dosage forms.

Although we do not wish to be bound by any particular theory or mechanism of action, it is believed that the treatment regimens of the present embodiment of the invention suppress the hypothalamic-pituitary-ovarian axis but do not cause hypoestrogenemia because of the exogenous estrogen component of the embodiment of the invention replaces the suppressed endogenous estrogen. It is believed that the combination of estrogen and progestin at a constant dosage suppresses endogenous hormonal fluctuations, as well as ovarian activity and the cyclic variations in the production of estrogen, progesterone, luteinizing hormone, and follicle-stimulating hormone.

The methods of the invention can be evaluated for their effect on cycle-related symptoms using, for example, psychometric scales that include a prospective daily symptoms chart or diary, such as the 17-item Penn Daily Symptom Report (DSR) to evaluate physical and psychological symptoms. Total score of the physical and psychological symptoms is computed. The 17-item Penn DSR was used to measure cycle-related symptoms in CRSS subjects who met predefined criteria for a subgroup with dysmenorrhea, a subgroup with moderate to severe cycle-related symptoms, and a subgroup with mild to moderate cycle-related symptoms.

### EXAMPLES

### Example 1

Cycle-Related Symptoms Study Methods

The cycle-related symptom study (CRSS) was a 3-month study that evaluated the effects of a continuous use regimen of progestin and estrogen on cycle-related symptoms. Cycle-related symptoms were evaluated among subgroups of female subjects with symptoms of: 1) dysmenorrhea or; 2) two groups of cycle-related symptoms which include the moderate to severe cycle-related symptoms subgroup, and the mild to moderate cycle-related symptoms subgroup. Subjects were to have a history of dysmenorrhea or symptoms of moderate to severe cycle-related symptoms and must have met the study definition of cycle-related symptoms during the screening menstrual cycle, with data collected prospectively on a validated cycle-related symptoms questionnaire, i.e., 17-item Penn Daily Symptom Report (DSR), which included a 5-point Likert scale used by subjects to rate the severity of symptoms. Each symptom was rated on a 5-point Likert scale as follows: 0=none; 1=minimal; 2=moderate (not affecting daily activities); 3=a lot (continuous, or interfering with activities); and 4=severe (overwhelming and/or preventing daily activities. The 17-item Penn DSR measured cycle-related symptoms associated with a woman's menstrual cycle, which include, but are not limited to, psychological symptoms (for example, mood change, irritability, anxiety, lack of concentration, or decrease in sexual desire) and physical symptoms (for example, dysmenorrhea, breast tenderness, bloating, fatigue, or food cravings).

Subjects in the CRSS who met the criteria were expected to complete the 17-item Penn DSR daily during the baseline cycle and during pill packs 1, 2, and 3 (28 daily doses per pill pack).

The Endicott Work Productivity Scale (EWPS) was a questionnaire administered to all subjects who qualified for the two cycle-related symptoms subgroups or dysmenorrhea subgroup of the CRSS and who received pay for work or did volunteer work. The subjects were to complete the EWPS on days 7, 14, 21, and 28 of the baseline screening cycle, and during pill packs 1, 2, and 3 (28 daily doses per pill pack).

The CRSS results showed that continuous-use regimen of progestin and estrogen to female subjects reduced cycle-related symptoms. The dosage of progestin (levonorgestrel, LNG: 90 μg) and estrogen (ethinyl estradiol, EE: 20 μg) in a continuous-use regimen to female subjects was effective in rapidly reducing cycle-related symptoms associated with menses. Treatment of women in the dysmenorrhea subgroup (n=259) showed that the LNG 90 μg/EE 20 μg continuous-use regimen was highly effective in reducing cramps during the second and third month of treatment (no significant effect was expected in the first month) and continuing throughout the treatment period. Treatment of women in the moderate to severe cycle-related symptoms subgroup (n=78) showed a reduction by more than 50% in a broad range of cycle-related symptoms, including moderate to severe cycle-related symptoms, starting during the first month of treatment and continuing throughout the treatment period. Treatment of women in the mild to moderate cycle-related symptoms subgroup (n=36) showed that the LNG 90 μg/EE 20 μg continuous-use regimen was effective in reducing most cycle-related symptoms in women with less severe cycle-related symptoms.

Dysmenorrhea Subgroup

A total of 259 subjects met the protocol-defined criteria for dysmenorrhea and were included in this subgroup analysis. Of these subjects, 233 had complete data for pill pack 1, a total of 224 had complete data for pill pack 2, and 199 had complete data for pill pack 3 (28 daily doses per pill pack).

The mean scores for cramps during pill pack 2 and pill pack 3 were decreased from baseline, indicating that the LNG 90 μg/EE 20 μg continuous-use regimen was highly effective in reducing cramps in this subgroup. Because subjects began pill pack 1 on the first day of menses, no significant effect of treatment on dysmenorrhea was expected during pill pack 1.

Based on the mean maximum cramps score reported during the first five days of the baseline cycle for each pill pack, the severity of dysmenorrhea, as reflected in the mean maximum cramps score, decreased with each pill pack. The decrease from baseline was greatest during pill pack 2 and pill pack 3. Because subjects began pill pack 1 on the first day of menses, no significant effect of treatment on the severity of dysmenorrhea was expected during pill pack 1.

Moderate to Severe Cycle-Related Symptoms Subgroup

A total of 78 subjects met the protocol-defined criteria for moderate to severe cycle-related symptoms symptoms. The moderate to severe cycle-related symptoms subgroup comprised subjects that had one or more moderate to severe cycle-related symptoms. Of those subjects, 70 had complete data for pill pack 1, a total of 64 had complete data for pill pack 2, and 56 had complete data for pill pack 3. Scores for moderate to severe cycle-related symptoms were reported for six premenstrual days (i.e., days 23 to 28) and six postmenstrual days (i.e., days 6 to 11), and were summarized as the mean score for symptoms separately and then collectively as the mean total score.

The mean total scores reported for premenstrual symptoms (days 23 to 28) during pill pack 1, pill pack 2, and pill pack 3 were decreased from baseline. The mean score for each of 17 individual premenstrual symptoms also decreased from baseline. Moderate to severe cycle-related symptoms was defined by the protocol as a premenstrual total score ≧80 and a post menstrual total score ≦50 on the 17-item Penn DSR at baseline

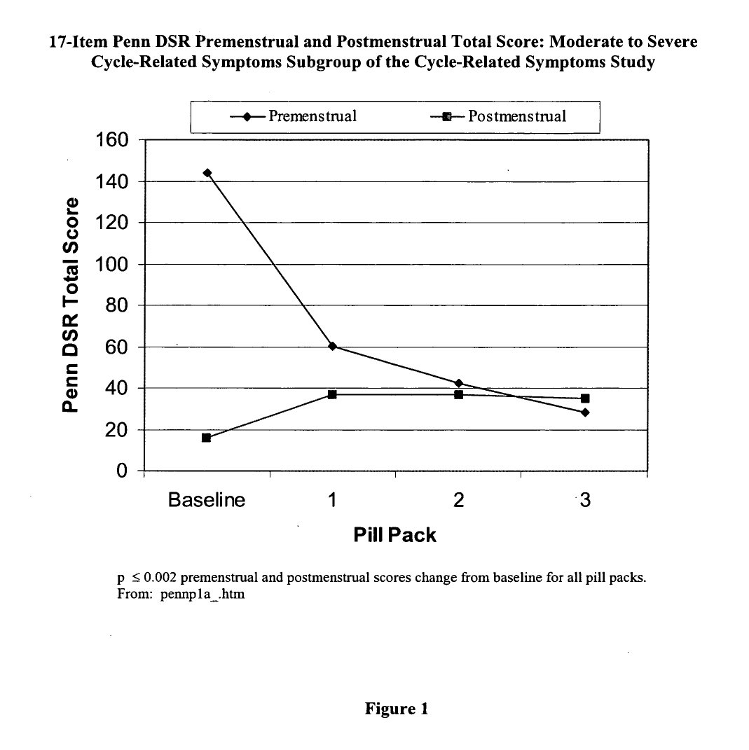

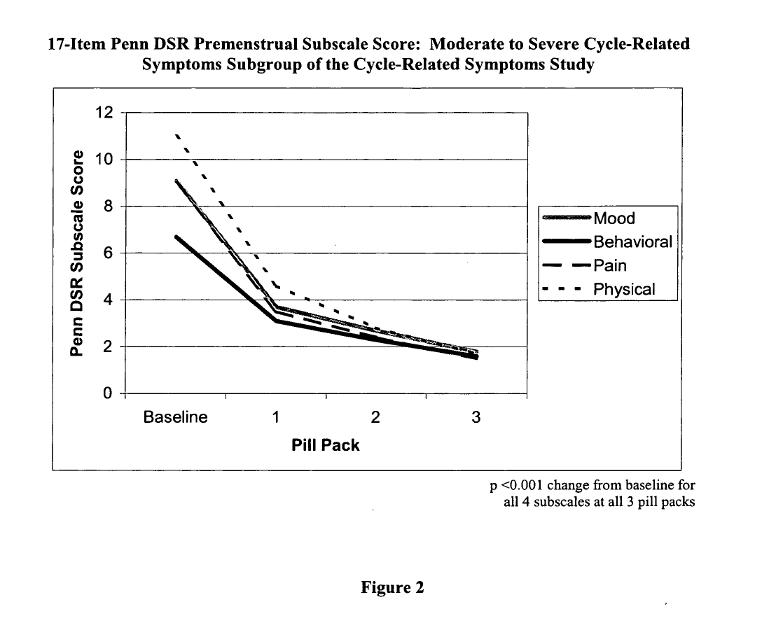

While mean total score for postmenstrual symptoms was slightly increased during pill pack 1, the mean premenstrual total score was essentially equivalent to the mean postmenstrual score in pill packs 2 and 3 (FIG. 1). In addition, the subscale scores for the moderate to severe cycle-related symptoms subgroup with symptoms related to mood, behavior, pain, or other physical symptoms decreased during pill pack 1, and further decreased during pill packs 2 and 3 (FIG. 2).

Mild to Moderate Cycle-Related Symptoms Subgroup

Subjects included in the mild to moderate cycle-related symptoms subgroup had symptoms less severe than those required for subjects in the moderate to severe cycle-related symptoms subgroup yet still had cycle-related symptoms as defined by the protocol (i.e., premenstrual score of ≧50 but ≦79 and postmenstrual score <50 on the 17-item Penn DSR). Thirty-six (36) subjects met the protocol-defined criteria. Of these subjects, 31 had complete data for pill pack 1, a total of 29 had complete data for pill pack 2, and 26 had complete data for pill pack 3.

Mean total and mean individual scores for cycle-related symptoms were reported for six premenstrual days (i.e., days 23 to 28) and six postmenstrual days (i.e., days 6 to 11). The mean total scores reported for premenstrual (days 23 to 28) symptoms during pill pack 1, pill pack 2, and pill pack 3 were decreased from baseline.

For individual items, the premenstrual scores were reduced from baseline during all three pill packs for the symptoms defined as fatigue, aches, irritability, mood swings, swelling, craving food, breast tenderness, and cramps. Individual items were significantly reduced from baseline during pill pack 1 only for symptoms defined as poor coordination, out of control, and nervous tension. Anxiety and insomnia were reduced from baseline during pill pack 1 and 2, but not pill pack 3. The symptoms defined as headache and confusion were not significantly reduced in pill pack 1, but were in pill pack 2 and/or 3. Depression and crying were not significantly reduced from baseline for any pill pack. The mean total postmenstrual score increased from baseline to pill pack 1 and had no further increase in pill packs 2 and 3. A significant increase in postmenstrual scores for some individual symptoms was observed in each of the pill packs.

Endicott Work Productivity Scale

The Endicott Work Productivity Scale (EWPS) total scores were evaluated for subjects in the dysmenorrhea subgroup both at baseline and at evaluation week 1 of each pill pack. The mean total EWPS score at baseline decreased by pill pack 1. Similar decreases were observed from baseline to week 1 of each subsequent pill pack.

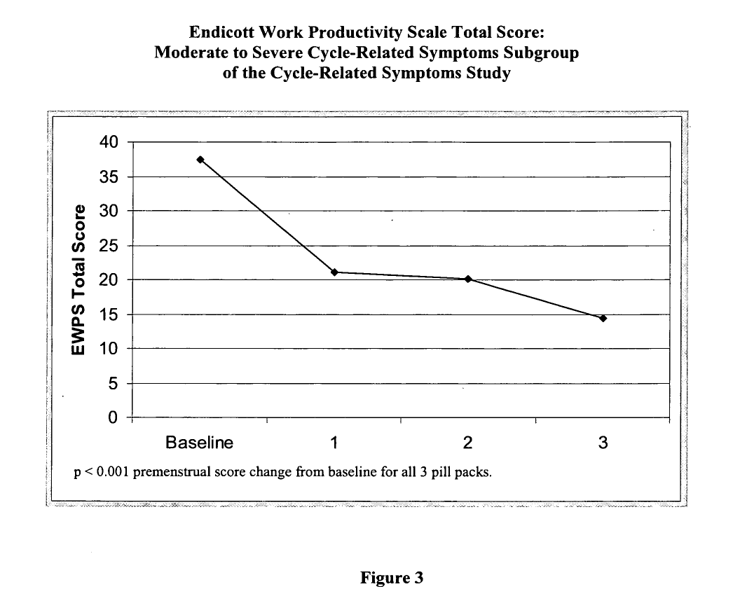

The EWPS total scores were evaluated for subjects in the moderate to severe cycle-related symptoms subgroup both at baseline and at evaluation week 4 of each pill pack (FIG. 3). For this subgroup, the mean total score at evaluation week 4 decreased from baseline to pill pack 1 and decreased further in pill packs 2 and 3. The mean total EWPS scores at evaluation week 4 of each pill pack were all decreased from baseline.

For the dysmenorrhea subgroup, the moderate to severe cycle-related symptoms subgroup, and the mild to moderate cycle-related symptoms subgroup, the EWPS scores were decreased from baseline over the appropriate evaluation week after pill pack 1 and were 53% and 37% of the baseline score, respectively, by pill pack 3, indicating improved work productivity. These results represented rapid improvements in work productivity that was evident as early as pill pack 1 and that continued to improve through pill pack 3.

The LNG 90 μg/EE 20 μg continuous-use regimen was effective in rapidly reducing cycle-related symptoms typically associated with menses. Results from the dysmenorrhea subgroup (n=259) showed that the LNG 90 μg/EE 20 μg continuous-use regimen was highly effective in reducing cramps by pill pack 2 (second 28-day pack), a benefit that continued through pill pack 3 (third 28-day pack). Results from the moderate to severe cycle-related symptoms subgroup (n=78) showed a reduction by more than 50% in a broad range of moderate to severe cycle-related symptoms by the end of pill pack 1 (first 28-day pack), and a reduction of 80% during pill pack 3 (third 28-day pack). Results from the mild to moderate cycle-related symptoms subgroup (n=36) showed that the LNG 90 μg/EE 20 μg continuous-use regimen was effective in reducing most cycle-related symptoms.

The reductions in cycle-related symptoms observed with the LNG 90 μg/EE 20 μg continuous-use regimen were consistent with improved work productivity among CRSS subjects who worked and/or volunteered (dysmenorrhea subgroup and moderate to severe cycle-related symptoms subgroup only). Among the subjects evaluated, the EWPS scores were significantly decreased from baseline after the first pill pack (first 28-day pack). These results represented a rapid improvement in work productivity that continued to improve through pill pack 3 (third 28-day pack).

What is claimed:

1. A method for treating a female subject having cycle-related symptoms comprising administering an effective amount of at least one progestin and at least one estrogen to said female subject, wherein said effective amount is administered daily for at least about 100 days.

2. The method of claim 1 wherein said female subject has one or more psychological or physical cycle-related symptoms and said effective amount is effective for treating said one or more psychological or physical symptoms.

3. The method of claim 2 wherein said female subject has dysmenorrhea and said effective amount is effective for treating dysmenorrhea.

4. The method of claim 2 wherein said female subject has moderate to severe cycle-related symptoms and said effective amount is effective for treating moderate to severe cycle-related symptoms.

5. The method of claim 2 wherein said female subject has mild to moderate cycle-related symptoms and said effective amount is effective for treating mild to moderate cycle-related symptoms.

6. The method of claim 1 wherein at least about 4 μg of levonorgestrel, or a progestin dosage of corresponding potency, is administered.

7. The method of claim 1 wherein from about 60 μg to about 110 μg of levonorgestrel, or a progestin dosage of corresponding potency, is administered.

8. The method of claim 1 wherein at least about 1 μg of ethinyl estradiol, or an estrogen dosage of corresponding potency, is administered.

9. The method of claim 1 wherein from about 15 μg to about 25 μg of ethinyl estradiol, or an estrogen dosage of corresponding potency, is administered.

10. The method of claim 1 wherein said at least one progestin is chlormadinone acetate, norethisterone acetate, cyproterone acetate, desogestrel, gestodene, drospirenone, etonorgestrel, norgestimate, norelgestromin, or levonorgestrel.

11. The method of claim 1 wherein said at least one estrogen is ethinyl estradiol, mestranol, estradiol, estriol, estrone, or estrane.

12. The method of claim 10 wherein said at least one progestin is levonorgestrel administered at a dosage not greater than 90 μg per day, or a progestin dosage of corresponding potency is administered.

13. The method of claim 11 wherein said at least one estrogen is ethinyl estradiol administered at a dosage not greater than 20 μg per day, or an estrogen dosage of corresponding potency is administered.

14. The method of claim 1 wherein said at least one estrogen is ethinyl estradiol administered at a dosage of approximately 20 μg per day, and said at least one progestin is levonorgestrel administered at a dosage of approximately 90 μg per day, or an estrogen dosage of corresponding potency and a progestin dosage of corresponding potency is administered.

15. The method of claim 1 wherein said effective amount is administered orally, transdermally, or via depot to the subject.

16. The method of claim 1 wherein said effective amount is administered daily for at least about 4 months.

17. The method of claim 1 wherein said effective amount is administered daily for at least about 6 months.

18. The method of claim 1 wherein said effective amount is administered daily for at least about 9 months.

19. The method of claim 1 wherein said effective amount is administered daily for at least about 12 months.

20. The method of claim 1 wherein said effective amount is administered in a dosage form.

21. The method of claim 1 wherein said dosage form is a tablet or capsule.

22. The method of claim 20 wherein said dosage form is administered orally, transdermally or via depot to the subject.

23. The method of claim 1 further comprising determining an extent to which said female subject has cycle-related symptoms.

24. The method of claim 23, further comprising determining an extent to which said female subject has dysmenorrhea.

25. The method of claim 23, further comprising determining an extent to which said female subject has moderate to severe cycle-related symptoms.

26. The method of claim 23 further comprising determining an extent to which said female subject has mild to moderate cycle-related symptoms.

27. The method of claim 23 wherein said determination is made before said administering step.

28. The method of claim 23 wherein said determination is made after said administering step.

29. A kit for treating a female subject having cycle-related symptoms comprising at least about 100 dosage forms that individually comprise at least one progestin and at least one estrogen.

30. The kit of claim 29 wherein each of said dosage forms comprise at least about 4 μg of levonorgestrel or a progestin dosage form of corresponding potency.

31. The kit of claim 29 wherein each of said dosage forms comprise from about 60 μg to about 110 μg of levonorgestrel or a progestin dosage form of corresponding potency.

32. The kit of claim 29 wherein each of said dosage forms comprise at least about 1 μg of ethinyl estradiol or an estrogen dosage form of corresponding potency.

33. The kit of claim 29 wherein each of said dosage forms comprise from about 15 μg to about 25 μg of ethinyl estradiol or an estrogen dosage form of corresponding potency.

34. The kit of claim 29 wherein said at least one progestin is progesterone, chlormadinone acetate, norethisterone acetate, cyprotherone acetate, desogestrel, drospirenone, etonorgestrel, norgestimate, norelgestromin, or levonorgestrel.

35. The kit of claim 29 wherein said at least one estrogen is ethinyl estradiol, mestranol, estradiol, estriol, estrone, or estrane.

36. The kit of claim 34 wherein each of said dosage forms comprise levonorgestrel in an amount no greater than 90 μg or a progestin dosage form of corresponding potency.

37. The kit of claim 35 wherein each of said dosage forms comprise ethinyl estradiol in an amount no greater than 20 μg or an estrogen dosage form of corresponding potency.

38. The kit of claim 29 wherein each of said dosage forms comprise approximately 20 μg ethinyl estradiol and approximately 90 μg levonorgestrel, or an estrogen dosage form and a progestin dosage form of corresponding potency.

39. The kit of claim 29 that comprises at least about 185 of said dosage forms.

40. The kit of claim 29 that comprises at least about 275 of said dosage forms.

41. The kit of claim 29 that comprises is at least about 365 of said dosage forms.

42. The kit of claim 29 wherein said dosage forms are tablets, capsules, or a combination thereof.
